(19) United States
(12) Patent Application Publication
(10) Pub. No.: US 2004/0068241 A1
(43) Pub. Date: Apr. 8, 2004

(54) Implantable medical device

(76) Inventor: Frank J. Fischer JR., Bloomington, IN (US)

Correspondence Address: BRINKS HOFER GILSON & LIONE, P.O. BOX 10395, CHICAGO, IL, 60611, US

(21) Appl. No.: 10/410,587

(22) Filed: Apr. 8, 2003

Related U.S. Application Data
(63) Continuation-in-part of application No. 08/868,518, filed on Jun. 4, 1997, now Pat. No. 6,599,275.
(60) Provisional application No. 60/018,924, filed on Jun. 4, 1996.

(51) Int. Cl. A61M 25/00

(52) U.S. Cl. 604/265; 424/424

(57) ABSTRACT

A medical device comprises first and second members and first and second therapeutic agents. The agents have different diffusion rates through at least one of the members, and are positioned within the medical device based on the diffusion rates. The therapeutic agent having the greater diffusion rate is positioned in the device at a location that is a greater distance from a treatment site than that of the location of the therapeutic agent having the lesser diffusion rate. Methods of making medical devices include placing two or more therapeutic agents within a device based on the diffusion rates of the agents through at least one portion of the device.

### REFERENCE TO RELATED APPLICATIONS

This application is a continuation-in-part of application Ser. No. 08/868,518, filed on Jun. 4, 1997, and entitled “Implantable Medical Device”, which claims the benefit of provisional application Serial No. 60/018,924, filed on Jun. 4, 1996.

### FEDERALLY SPONSORED RESEARCH OR DEVELOPMENT

This application was funded in part under the following research grants and/or contracts: NASA Grant No. NAG 8-1471 and NSF DMR 94-53446. The United States government may have rights in this invention.

### BACKGROUND OF THE INVENTION

The present invention relates generally to medical devices, and more particularly to medical devices that are at least partially implantable into a human or veterinary patient.

It has become common to treat a variety of medical conditions by introducing an implantable medical device partly or completely into a portion of the body, such as a vessel. For example, many treatments of the vascular system entail the introduction of medical devices, such as stents, catheters, balloons, wire guides, cannulae, and the like, into a vessel, such as an artery or vein. The device utilized may serve a variety of purposes, such as maintaining vessel patency, providing access to a body portion, and delivering one or more therapeutics.

During introduction and/or implantation of these devices, however, adverse affects can occur. For example, the vessel walls can be disturbed or injured during navigation of the device through the vessel. As a result, clot formation or thrombosis can occur at the site of injury, which may cause stenosis or occlusion of the vessel. Moreover, if the device is left within the patient for an extended period of time, a thrombus often forms on the device itself, which may also lead to stenosis or occlusion of the vessel. These conditions may place the patient at risk of a variety of complications, including heart attack, pulmonary embolism, and stroke. Thus, the use of such a medical device can include the risk of causing precisely the problems that its use was intended to ameliorate.

Implantable medical devices also present an opportunity for the establishment of infection. Microorganisms may colonize the device and establish an infection at the implant site, which may cause injury or illness and may even destroy the functionality of the device. The risk of infection is particularly acute for partially implanted medical devices, percutaneously introduced into the vascular system of a patient for long term use, such as hemodialysis and drug infusion catheters. These devices are exposed to both the external and internal environments, providing a link between these two very different environments. Microorganisms can use the device to gain access to the internal environment, ultimately colonizing and possibly establishing an infection. Indeed, the occurrence of infection with indwelling catheters is a common problem that can necessitate repeated removal and replacement of catheters, in addition to treatment of infections.

The art contains many examples of devices adapted to inhibit or prevent such infections. For example, U.S. Pat. No. 4,677,143 to Lavrin describes an antimicrobial coating placed on the exterior of a medical device, such as a catheter. Also, U.S. Pat. No. 3,598,127 to Wepsic describes a device with an antimicrobial placed as a powder in the device and surrounded by a permeable layer. Furthermore, devices are known that include more than one therapeutic agent. For example, U.S. Pat. No. 5,820,607 to Tcholakia describes a layered catheter that includes an intermediate layer surrounded by a permeable layer. The intermediate layer can include multiple therapeutic agents. Also, U.S. Pat. No. 4,999,210 to Solomon describes a layered device that can include different therapeutic agents in different layers.

In some circumstances, it is desirable to administer combination therapy to a patient. In combination therapy, two or more therapeutic agents are delivered to a patient to achieve a desired result. The agents may act in concert, have the same, similar, or different targets, and have the same, similar, or different mechanisms of action. Furthermore, the agents can be delivered at substantially the same time, sequentially, or completely independently of each other. The method and timing of delivery of the agents depends on several factors, including the agents utilized, the targets of their action, and the condition being treated. One widely accepted example of combination therapy is the use of two or more antibiotics to treat a microbial infection. Commonly, rifampin and minocycline are used together.

While the devices in the prior art, such as those listed above, may provide some benefit, the art fails to teach a medical device adapted to optimize the delivery of multiple therapeutic agents in order to take full advantage of the benefits of combination therapy. For example, when rifampin and minocycline are administered systemically for combination therapy, such as by ingestion of a solid dosage form or by injection, both therapeutic agents are available at a treatment site at substantially identical times. When delivered locally by a medical device, however, the device presents physical limitations on delivery, which may affect the availability of the agents at the treatment site. For example, if the therapeutic agents must diffuse through a portion of the device in order to arrive at the treatment site, the ability of each agent to diffuse through the portion will affect the availability of the agent at the treatment site. When two different therapeutic agents are utilized, they may have different diffusion abilities and rates, creating differences in the availability of the individual therapeutic agents at the treatment site. This may affect the effectiveness of the combination therapy. For example, if one therapeutic agent is not available at the site, or is available only in a suboptimum amount or at an undesired time, the benefits of combination therapy may be diminished or lost.

### SUMMARY OF THE INVENTION

The present invention provides a medical device that includes first and second therapeutic agents. The agents are positioned in different areas or locations of the device based on their respective diffusion rates through a portion of the device. This configuration provides control over the delivery of multiple therapeutic agents through the medical device, making the device particularly well suited for the delivery of combination therapy, such as therapy utilizing multiple anti-microbial agents.

In one embodiment, a medical device according to the invention includes first and second members. Preferably, the members comprise first and second elongated members. The first and second elongated members are coaxial, and the second elongated member is spaced from the first elongated member to define an intermediate region between the members. A first therapeutic agent is disposed within the intermediate region and has a first diffusion rate through the second elongated member. A second therapeutic agent is associated with the second elongated member and has a second diffusion rate through the second elongated member. The first diffusion rate is greater than the second diffusion rate.

The invention is particularly well suited for use in catheters and other cannula-type medical devices. Accordingly, in one embodiment, a medical device according to the invention comprises an outer tube defining a lumen and comprising a first therapeutic agent, and an inner tube disposed within the lumen to define an intermediate region between the outer and inner tubes. A therapeutic composition is disposed within the intermediate region, and comprises the second therapeutic agent. The first therapeutic agent has a first diffusion rate through the outer tube and the second therapeutic agent has a second diffusion rate through the outer tube. The second diffusion rate is greater than the first diffusion rate. The therapeutic composition may further comprise the first therapeutic agent.

The invention also includes methods of making a medical device. The methods include placing multiple therapeutic agents in different areas or locations of a medical device based on the respective diffusion rates of the agents through a portion of the device. One method according to the invention comprises providing first and second elongated members and providing first and second therapeutic agents. The method further comprises determining the diffusion rates of the first and second therapeutic agents through the second elongated member, and determining which of the first and second therapeutic agents has the greater diffusion rate. The method further comprises forming an intermediate region between the first and second elongated members, and placing the one of the first and second therapeutic agents having the greater diffusion rate in the intermediate region. The method further comprises associating the one of the first and second therapeutic agents having the lesser diffusion rate with the second elongated member.

Another method of making a medical device according to the present invention comprises providing first and second therapeutic agents and determining the diffusion rates of the agents through a base material. The method further comprises forming a first elongated member of the base material containing the one of the first and second therapeutic agents having the lesser of the diffusion rates and placing a second elongated member adjacent the first elongated member to define an intermediate region between the first and second elongated members. The method further comprises placing the one of the first and second therapeutic agents having the greater diffusion rate in the intermediate region.

Another method of making a medical device according to the present invention comprises providing first and second therapeutic agents. The method further comprises selecting a base material through which the first therapeutic agent has a first diffusion rate and the second therapeutic agent has a second diffusion rate that is greater than the first diffusion rate. The method further comprises forming a first elongated member of the base material and the first therapeutic agent, and placing a second elongated member adjacent the first elongated member to define an intermediate region between the first and second elongated members. The method further comprises placing the second therapeutic agent in the intermediate region.

### BRIEF DESCRIPTION OF THE DRAWINGS

FIG. 1 is a cross-sectional view of a medical device according to one embodiment of the invention.

FIG. 2 is a cross-sectional view of a medical device according to another embodiment of the invention.

FIG. 3 is a cross-sectional view of a medical device according to another embodiment of the invention.

FIG. 4 is a perspective view, partially broken away, of a catheter according to one embodiment of the invention.

FIG. 5 is a cross-sectional view of a medical device according to another embodiment of the invention.

FIG. 6 is a cross-sectional view of a medical device according to another embodiment of the invention.

### DETAILED DESCRIPTION OF THE INVENTION

The present invention provides a medical device with first and second therapeutic agents. The agents are positioned in the device based on their respective diffusion rates through a portion of the device. Preferably, the device comprises first and second elongated members. The second elongated member is spaced from the first elongated member to define an intermediate region between the first and second elongated members. At least the one of the first and second therapeutic agents having the greater diffusion rate is placed within the intermediate region. The one of the first and second therapeutic agents having the lesser diffusion rate is associated with at least one of the first and second elongated members. To be associated with an elongated member, a therapeutic agent can be applied to a surface of the member, such as by coating, dispersed in the base material of the member, e.g., bulk distribution, or both. Indeed, any suitable technique for placing a therapeutic agent in, on, or near a medical device for delivery through the device can be utilized.

The invention is suitable for any medical device in which first and second therapeutic agents can be positioned. The invention is particularly well suited for devices used for the delivery of first and second therapeutic agents. Examples of types of devices that can be made in accordance with the present invention include stents, catheters, cannulae, balloons, and bladders. The device need only be at least partially implantable in a patient.

The first and second elongated members can be made from any suitable material. The material need only be acceptable for use in a medical device, i.e., biocompatible and acceptable for the intended use of the device. Preferably, the material is able to have one or more therapeutic agents associated with it. Examples of suitable materials include materials commonly used in medical devices, such as polymers, copolymers, plastics, and metals. The material chosen will depend on several factors, including the intended use of the device, the therapeutic agents that will be used in the device, the ability of the material to have one or more of the agents associated with it, the permeability of the material to the therapeutic agents, and the ability of the material to be formed into members permeable to the therapeutic agents.

Silicone is a preferred material for use in one or all of the elongated members of the medical devices according to the present invention. Silicone is preferred for several reasons, including its widespread use in a variety of medical devices, its known biocompatibility, its permeability to numerous sizes, shapes, and types of therapeutic agents, and its ability to associate with therapeutic agents by coating, bulk distribution, and combinations of these approaches. Furthermore, silicone is particularly preferred because it enables the use of bulk distribution methods involving relatively low temperatures, as compared to the higher temperatures needed in methods using thermoplastics and other materials. The use of these relatively low temperatures minimizes damage to the therapeutic agents being distributed within the material. Also, silicone is readily available from a variety of commercial sources in various forms, including powder form which can be readily used in bulk distribution methods.

Of course, multiple materials can be used in the medical devices according to the present invention. For example, the first and second elongated members can be formed of different materials. Also, the intermediate region between the first and second elongated members can comprise a layer of material, such as a third elongated member, which can be formed of a material different than that of one or both of the first and second elongated members.

A wide variety of therapeutic agents can be utilized in the present invention. Examples of suitable types of therapeutic agents include antimicrobials, antivirals, antiproliferatives, antithrombotics, antimitotics, proteins, nucleic acids, carbohydrates, conjugates, small molecules, and antibodies.

The actual types of agents chosen will depend upon the clinical situation being treated or addressed by the medical device of the invention. The therapeutic agents can be of the same or different types.

Two or more therapeutic agents are utilized in the medical devices of the invention. The difference between the therapeutic agents need only be a detectable difference in the diffusion rates of the agents through a portion of the medical device, as described below. Thus, if a suitable difference in diffusion rates exist, two or more derivatives of a therapeutic agent can be utilized. Also, a therapeutic agent and one or more derivatives of the agent can be used. Of course, two completely different therapeutic agents can also be used, so long as a suitable difference in diffusion rates exist between the agents.

Preferably, the therapeutic agents are agents conventionally used in combination therapy. Particularly preferable, the therapeutic agents are agents commonly used in the treatment, inhibition, and/or prevention of microbial infections. Rifampin and minocycline are a particularly preferred pair of therapeutic agents for use in the medical devices according to the present invention.

The first and second therapeutic agents have different diffusion rates through a portion of the medical device. That is, the agents have different diffusion rates through the same portion of the device. As used herein, the term ‘diffusion rate’ refers to the rate, i.e., distance/time, at which an agent travels through a permeable material. The diffusion rate of each therapeutic agent can be determined analytically by any suitable procedure. For example, zone of inhibition tests, known to those skilled in the art, can be used to determine the rate at which an antimicrobial diffuses through a section of material, such as silicone, polyurethane, and polyethylene. Relative diffusion rates of two or more therapeutics can be determined by analyzing each therapeutic under the same test conditions.

A therapeutic agent with a greater diffusion rate travels a further distance through the material per unit time than an agent with a lesser diffusion rate. That is, the agent with the greater diffusion rate travels faster than the agent with the lesser diffusion rate. Stated differently, the therapeutic agent with the lesser diffusion rate travels slower through the material than the agent with the greater diffusion rate.

Diffusion rates will vary based on a variety of factors, including the material, the size and form of the agent, and the presence of other entities, such as other components, materials, and/or compositions, within the material, such as other therapeutic agents. Thus, the therapeutic agents and the materials for the device can be selected based on these and other parameters to optimize the delivery of the agents to one or more treatment sites, such as the tissue surrounding the exterior of the device and the area within an interior of the device. Given a particular material from which at least a portion of a medical device will be fabricated, two or more therapeutic agents can be selected based upon their diffusion rates through the material. The therapeutic agents are then arranged in the medical device based upon their diffusion rates. Also, given two or more specific therapeutic agents, a material can be selected for use in the medical device based upon the diffusion rates of the therapeutic agents through the material. For example, if it is known that two particular therapeutic agents are desirable for use in a particular combination therapy, a material can be selected based upon the permeability of the material and the diffusion rates of these agents through the material.

As indicated above, the therapeutic agents are arranged in the medical device according to their diffusion rates through at least a portion of the medical device. The final configuration and/or location of the therapeutic agents in the device will depend upon the desired availability of the agents in the combination therapy. For example, in some combination therapies, it may be desirable to have the therapeutic agents available at a particular treatment site at substantially the same time. For these therapies, the therapeutic agents are arranged in the medical device according to their diffusion rates in such a manner that the agents arrive at a treatment site at substantially the same time. Thus, the agent with the greater diffusion rate will be located a greater distance from the treatment site, while the agent with the lesser diffusion rate will be located a smaller distance from the treatment site. In other combination therapies, it may be desirable to have the first therapeutic agent arrive at a treatment site first, and subsequently have the second therapeutic agent arrive at the treatment site. In these instances, the therapeutic agents are arranged in the medical device according to their diffusion rates such that the first therapeutic agent arrives first, and the second therapeutic agent arrives second.

FIG. 1 illustrates a cross sectional view of a medical device according to one embodiment of the invention. While the illustrated device is a catheter, it is to be understood, as indicated above, that the invention can be applied to a wide variety of medical device types.

As illustrated in the figure, the device 10 includes first 12 and second 14 elongated members. The first elongated member 12 has an outer surface 16 and the second elongated member 14 has an inner surface 18. The second elongated member 14 encircles but is spaced from the first elongated member 12 to define an intermediate region 20 between the members 12, 14. More specifically, the intermediate region 20 is formed between the outer surface 16 of the first elongated member 12 and the inner surface 18 of the second elongated member 14.

In this embodiment, the intermediate region 20 comprises an annular space between the first 12 and second 14 elongated members. In other embodiments, the size and configuration of the intermediate region will depend on the configurations of the first 12 and second 14 elongated members, and the spatial relationship between the members 12, 14.

The medical device 10 further includes first 22 and second 24 therapeutic agents. The terms “first” and “second” therapeutic agents each refer to individual agents of a pair of agents. The agents differ in at least their diffusion rates and their location(s) in the medical device. In some embodiments, the first therapeutic agent will have a greater diffusion rate, while the second therapeutic agent will have the greater diffusion rate in other embodiments. In all embodiments, though, the agent with the greater diffusion rate is positioned a greater distance from a treatment site than the agent with the lesser diffusion rate. Of course, additional, i.e., third, fourth, etc., therapeutic agents can also be included in the devices according to the present invention.

The first therapeutic agent 22 is disposed in the intermediate region 20. In this embodiment, because the intermediate region 20 comprises a space between the first 12 and second 14 elongated members, the first therapeutic agent 22 is preferably in solid, e.g., powder, or liquid or gel form and is disposed in the space of the intermediate region 20. In this embodiment, the first therapeutic agent 22 is the one of the first 22 and second 24 agents having a greater diffusion rate. Preferably, the diffusion rates of the first 22 and second 24 therapeutic agents are determined relative to the second elongated member 14. That is, the diffusion rates preferably define the respective rates at which the therapeutic agents 22, 24 diffuse through the second elongated member 14. The diffusion rate of the first therapeutic agent through all or a portion of the intermediate region may be factored into the diffusion rates, their comparison, and the arrangement of the agents in the device.

The second therapeutic agent 24 is associated with at least one of the first 12 and second 14 elongated members. Thus, the second therapeutic agent 24 can be associated with the first elongated member 12, the second elongated member 14, or both. Furthermore, the second therapeutic agent can be associated with either of the members 12, 14 by surface coating or bulk distribution. The final positioning of the second therapeutic agent 24 in the device 10 will depend on the delivery modes desired in the combination therapy. For example, in the catheter illustrated in FIG. 1, it may be desirable to deliver one or both of the therapeutic agents 22, 24 to the tissue surrounding the exterior of the second elongated member 14 as well as to the interior lumen of the device 10. In this case, it may be desirable to associate the second therapeutic agent 24 with both the first 12 and second 14 elongated members. The second therapeutic agent 24 can also be disposed in the intermediate region 20 with the first therapeutic agent 22. This may allow for an extended delivery of the second therapeutic agent 24.

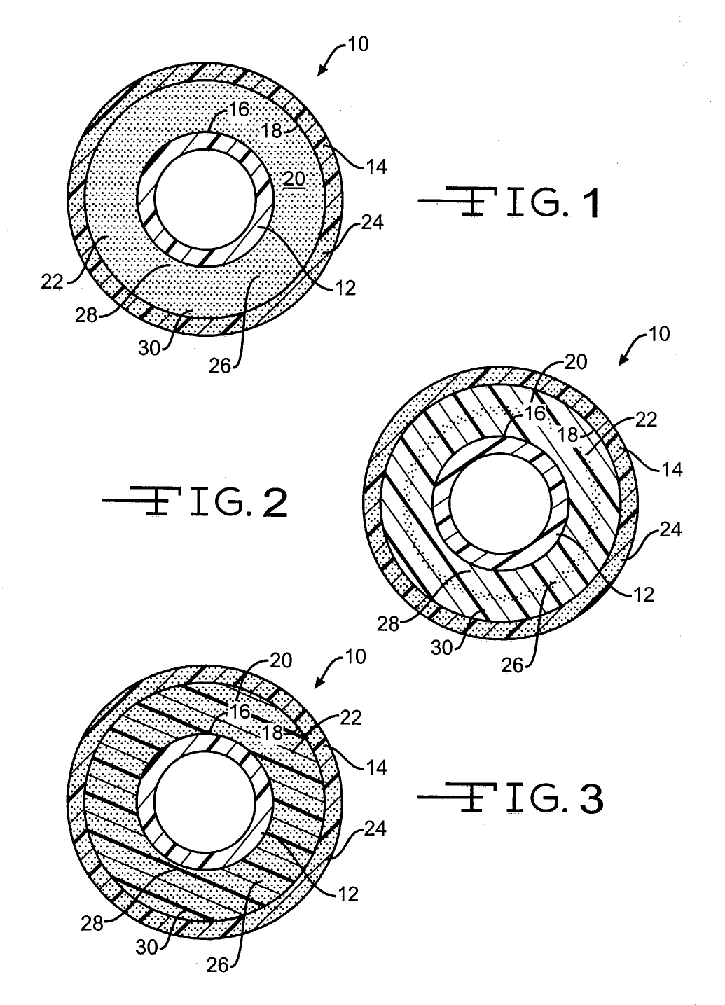

The intermediate region 20 has a center 26 and first 28 and second 30 outer edges. The first 28 and second 30 outer edges are adjacent the first 12 and second 14 elongated members, respectively. As illustrated in FIG. 1, the first therapeutic agent 22 can be substantially evenly distributed in the intermediate region 20 such that approximately equal concentrations of the therapeutic agent 22 exist at the center 26 and outer edges 28, 30. Alternatively, as illustrated in FIG. 2, the first therapeutic agent 22 can be distributed in the intermediate region 20 such that a greater concentration of the therapeutic agent 22 is in the center 26 than in the first 28 and second 30 outer edges. In this embodiment, the second therapeutic agent 24, if present in the intermediate region 20, can be substantially evenly distributed, or can be distributed in a similar manner as the first therapeutic agent 22, i.e., a greater concentration in the center. Also, the second therapeutic agent 24 can be distributed such that a greater concentration of the agent 24 exists in one or more of the outer edges 28, 30. The exact placement and distribution of the second therapeutic agent 24 in the intermediate region 20, if desired, can be optimized to control the diffusion of the first therapeutic agent 22 from the intermediate region 20 and through one or both of the elongated members 12, 14.

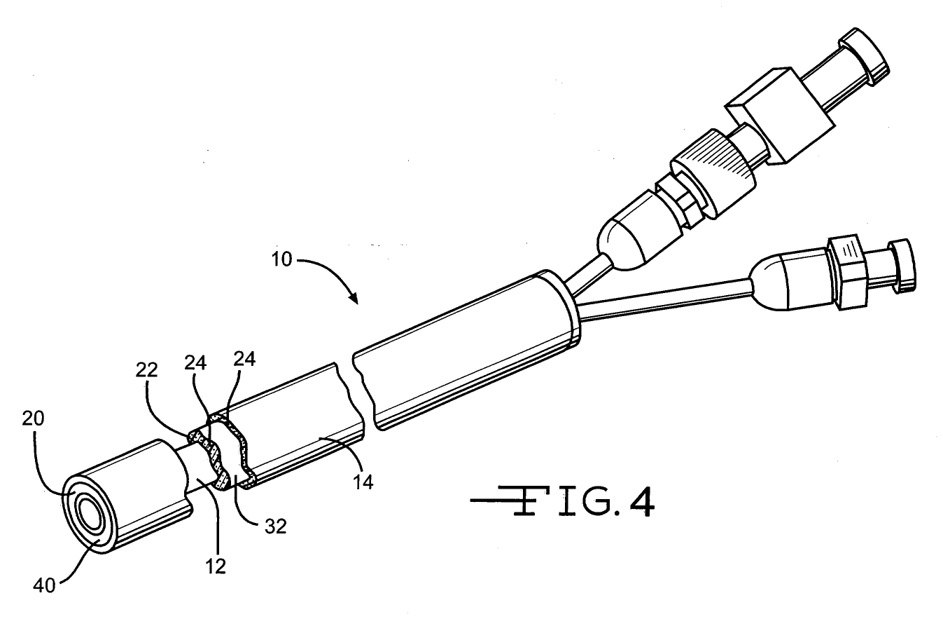

In another embodiment, illustrated in FIG. 3, the intermediate region 20 comprises a base material. Preferably, the intermediate region comprises a third elongated member 32 disposed between the first 12 and second 14 elongated members. In this embodiment, the first therapeutic agent 22 is associated with the third elongated member 32. Again, as indicated above, this can be accomplished by surface coating, bulk distribution, or both. Furthermore, the therapeutic agent 22 can be distributed in the third elongated member 32 in any suitable manner, including substantially uniform distribution and any other suitable distribution pattern, such as those described above. Preferably, the first therapeutic agent 22 is dispersed in the base material of the third elongated member in a substantially uniform manner.

As illustrated in FIG. 4, the device 10 preferably comprises a catheter. In this embodiment, the second elongated member 14 is a tube defining an inner passageway 40 or lumen. The first elongated member 12 is disposed in the passageway 40. Preferably, the first elongated member 12 also is a tube, and is preferably disposed coaxially within the inner passageway of the second elongated member 14 such that the intermediate region 20 is formed between the first 12 and second 14 elongated members. As indicated above, the intermediate region 20 can be an annular space or base material, such as a third elongated member 32 disposed between the first 12 and second 14 elongated members.

The elongated members and even the intermediate tube, if present, can include structural features that facilitate placement and/or replenishment of the therapeutic agent(s) present in the intermediate region. In this embodiment, the medical device can be recharged with therapeutic agent(s) one or more times when appropriate, such as when the concentration of one or more agents within the intermediate region becomes low or below a threshold value. This can facilitate continuation of the combination therapy over time.

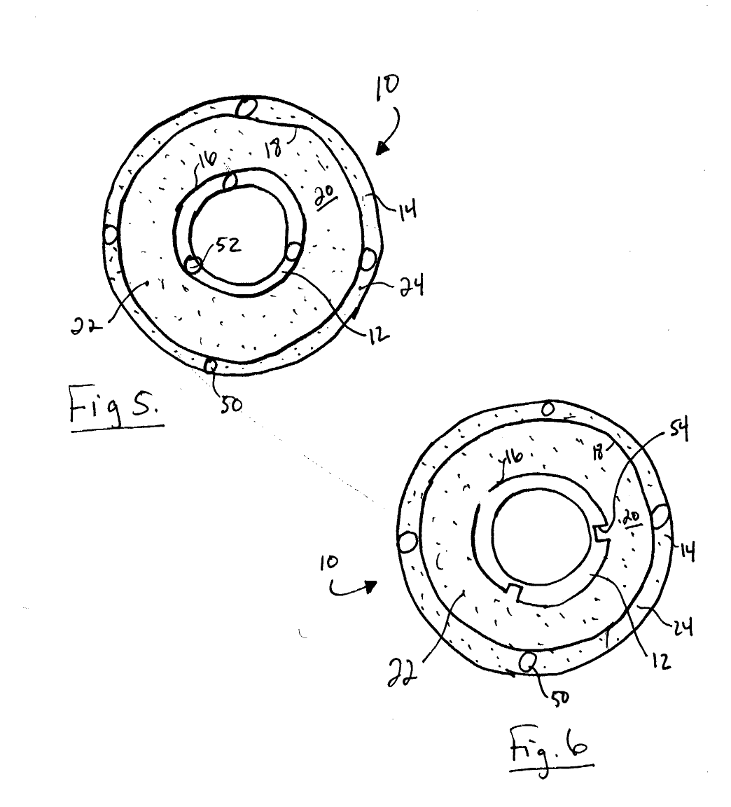

For example, one or more of the elongated members may define one or more channels or lumens in the wall of the member. The channel or lumen can extend from a distal portion of the device to a proximal portion of the device, allowing introduction of additional therapeutic agent(s) into the device. FIG. 5 illustrates a cross-sectional view of a medical device according to this embodiment of the invention. Each of the elongated members 12, 14 include lumens 50, 52, in the thickness of the respective wall. The lumens 50,52 allow for the introduction of a therapeutic, such as a recharging supply of the agent, by introduction of the agent into the lumen 50,52. The agents 50,52 are preferably introduced at a portion of the device that remains external to the body.

As illustrated in FIG. 5, each elongated member 12,14 preferably includes a plurality of lumens 50,52, which are preferably equidistant from each other. Of course, the specific number and location of the lumens will depend on numerous factors, including the agents used, their diffusion rates through the members 12,14, and the desired frequency of recharging.

FIG. 6 illustrates an alternative embodiment in which channels 54 are used in one of the elongated members 12. A channel 54 may be desireable, as opposed to a lumen 52, when a more rapid recharging of the appropriate agent is needed, because diffusion through the material of the member 12 is reduced. Any suitable combination of channels and/or lumens can be used in the devices of the present invention.

The present invention also provides methods of fabricating medical devices. In the methods according to the invention, one of at least two therapeutic agents having the greatest diffusion rate through a portion of the device is placed in an intermediate region between first and second members. A second therapeutic agent with a lesser diffusion rate is associated with at least one of the first and second members. Similar to the medical devices according to the invention, the methods of the invention preferably utilize elongated members, such as tube members.

In one method, the therapeutic agents and elongated members are provided, and diffusion rates are determined accordingly. This method comprises providing a first elongated member, providing a second elongated member, providing a first therapeutic agent, and providing a second therapeutic agent. The method further comprises determining diffusion rates of the first and second therapeutic agents through the second elongated member and determining which of the first and second therapeutic agents has the greater of the diffusion rates. The method further comprises forming an intermediate region between the first and second elongated members, and placing the one of the first and second therapeutic agents having the greater of the diffusion rates in the intermediate region. The method further comprises associating the one of the first and second therapeutic agents having the lesser of the diffusion rates with the second elongated member.

A second method according to the invention comprises providing first and second therapeutic agents and determining diffusion rates of the first and second therapeutic agents through a base material. The method further comprises forming a first elongated member of the base material containing the one of the first and second therapeutic agents having the lesser diffusion rate and placing a second elongated member adjacent the first elongated member to form an intermediate region between the first and second elongated members. The method further comprises placing the one of the first and second therapeutic agents having the greater diffusion rate in the intermediate region.

A third method according to the invention comprises providing first and second therapeutic agents and selecting a base material through which the first therapeutic agent has a first diffusion rate and the second therapeutic agent has a second diffusion rate greater than the first diffusion rate. The method further comprises forming a first elongated member of the base material and the first therapeutic agent. The method further comprises placing a second elongated member adjacent the first elongated member to form an intermediate region between the first and second elongated members and placing the second therapeutic agent in the intermediate region.

The foregoing disclosure includes the best mode devised by the inventor for practicing the invention. It is apparent, however, that several variations of the invention may be apparent to those of ordinary skill in the relevant art. Inasmuch as the disclosure is intended to enable one skilled in the pertinent art to make and use the invention, it should not be construed to be limited thereby, but rather should be construed to include such variations. Accordingly, the invention should be limited only by the spirit and scope of the claims.

### EXAMPLE

In one embodiment, a medical device according to the present invention comprises a catheter with nested tube members. An inner tube member is disposed in the lumen of an outer tube member, and an intermediate tube member is disposed between the inner and outer tube members. All members are positioned concentrically.

The intermediate member comprises a silicone tube with a rifampin/minocycline mixture evenly distributed throughout the member. The rifampin/minocycline mixture is added to a melt of the silicone material, and the material/mixture is then extruded into a tube shape. The rifampin/minocycline mixture is added to the silicone as a powder at 7% by weight of the silicone. The rifampin/minocycline mixture is a 50:50 weight percent mixture of the two therapeutic agents. Minocycline has a lower diffusion rate through silicone than rifampin. Accordingly, minocycline is also bulk distributed in the inner and outer tube members. The tubes are all extruded by a triple extrusion process.

The outer tube member is approximately 0.125″ in diameter with a wall thickness of approximately 0.007″. The inner tube member has an inner diameter of approximately 0.062″ and a wall thickness of approximately 0.007″. The intermediate member has a thickness of approximately 0.017″. The overall wall thickness of the device is approximately 0.031″.

The silicone used in the members has a diameter in a range of between about 30 and about 90 on the Share A Hardness Scale.

I claim:

1. A medical device for at least partial implantation in a patient, comprising:
a first elongated member;
a second elongated member spaced from the first elongated member to define an intermediate region between the first and second elongated members;
a first therapeutic agent disposed within the intermediate region, the first therapeutic agent having a first diffusion rate through the second elongated member; and
a second therapeutic agent associated with the second elongated member, the second therapeutic agent having a second diffusion rate through the second elongated member;
wherein the first diffusion rate is greater than the second diffusion rate.

2. The medical device of claim 1, wherein the second therapeutic agent is disposed within the intermediate region in addition to being associated with the second elongated member.

3. The medical device of claim 2, wherein the intermediate region has a center, a first outer edge adjacent the first elongated member, and a second outer edge adjacent the second elongated member; and
wherein a greater concentration of the first therapeutic agent is in the center of the intermediate region than in the first and second outer edges of the intermediate region.

4. The medical device of claim 3, wherein a greater concentration of the second therapeutic agent is in one of the first and second outer edges than in the center of the intermediate region.

5. The medical device of claim 2, wherein the second therapeutic agent is associated with the first elongated member in addition to being disposed in the intermediate region and associated with the second elongated member.

6. The medical device of claim 1, wherein the second therapeutic agent is associated with the first elongated member in addition to being associated with the second elongated member.

7. The medical device of claim 1, wherein the intermediate region comprises a third elongated member disposed between the first and second elongated members.

8. The medical device of claim 7, wherein the third elongated member comprises base material; and
wherein the first therapeutic agent is dispersed in the base material.

9. The medical device of claim 1, wherein the second elongated member comprises a tube defining an inner passageway.

10. The medical device of claim 9, wherein the first elongated member is disposed within the inner passageway.

11. The medical device of claim 1, wherein the second elongated member comprises base material; and
wherein the second therapeutic agent is dispersed in the base material.

12. The medical device of claim 11, wherein the second therapeutic agent is associated with the first elongated member in addition to being dispersed in the base material of the second elongated member.

13. The medical device of claim 12, wherein the first elongated member comprises base material; and
wherein the second therapeutic agent is dispersed in the base material of the first elongated member in addition to being dispersed in the base material of the second elongated member.

14. The medical device of claim 1, wherein at least one of the first and second therapeutic agents is an antibiotic.

15. The medical device of claim 14, wherein the first therapeutic agent comprises rifampin.

16. The medical device of claim 15, wherein the second therapeutic agent comprises minocycline.

17. A medical device for at least partial implantation in a patient, comprising:
an outer tube defining a lumen and comprising a first therapeutic agent;
an inner tube disposed within the lumen to define an intermediate region between the outer and inner tubes; and
a therapeutic composition disposed within the intermediate region, comprising the first therapeutic agent and a second therapeutic agent;
wherein the first therapeutic agent has a first diffusion rate through the outer tube and the second therapeutic agent has a second diffusion rate through the outer tube that is greater than the first diffusion rate.

18. The medical device of claim 17, wherein the intermediate region comprises an intermediate tube disposed between the inner and outer tubes.

19. The medical device of claim 18, wherein the intermediate tube comprises base material; and
wherein the therapeutic composition is dispersed in the base material.

20. The medical device of claim 19, wherein the intermediate region has a center, a first outer edge adjacent the outer tube, and a second outer edge adjacent the inner tube; and
wherein a greater concentration of the second therapeutic agent is in the center of the intermediate region than in the first and second outer edges of the intermediate region.

21. The medical device of claim 20, wherein a greater concentration of the first therapeutic agent is in one of the first and second outer edges than in the center of the intermediate region.

22. The medical device of claim 19, wherein at least one of the first and second therapeutic agents is an antibiotic.

23. The medical device of claim 22, wherein the first therapeutic agent comprises minocycline.

24. The medical device of claim 23, wherein the second therapeutic agent comprises rifampin.

25. A medical device for at least partial implantation, the medical device having multiple portions and first and second therapeutic agents positioned in the multiple portions based on respective diffusion rates of the agents through a portion of the device.

26. The medical device of claim 25, wherein the portion of the device comprises silicone.

27. The medical device of claim 25, wherein at least one of the first and second therapeutic agents is an antibiotic.

28. The medical device of claim 27, wherein the first therapeutic agent comprises rifampin.

29. The medical device of claim 28, wherein the second therapeutic agent comprises minocycline.

30. A method of making a medical device, comprising:
providing a first elongated member;
providing a second elongated member;
providing a first therapeutic agent;
providing a second therapeutic agent;
determining diffusion rates of the first and second therapeutic agents through the second elongated member;
determining which of the first and second therapeutic agents has the greater of the diffusion rates;
forming an intermediate region between the first and second elongated members;
placing the one of the first and second therapeutic agents having the greater of the diffusion rates in the intermediate region; and
associating the one of the first and second therapeutic agents having the lesser of the diffusion rates with the second elongated member.

31. A method of making a medical device, comprising:
providing first and second therapeutic agents;
determining diffusion rates of the fist and second therapeutic agents through a base material;
forming a first elongated member of the base material containing the one of the first and second therapeutic agents having the lesser diffusion rate;
placing a second elongated member adjacent the first elongated member to form an intermediate region between the first and second elongated members; and
placing the one of the first and second therapeutic agents having the greater diffusion rate in the intermediate region.

32. A method of making a medical device, comprising:
providing first and second therapeutic agents;
selecting a base material through which the first therapeutic agent has a first diffusion rate and the second therapeutic agent has a second diffusion rate greater than the first diffusion rate;
forming a first elongated member of the base material and the first therapeutic agent;
placing a second elongated member adjacent the first elongated member to form an intermediate region between the first and second elongated members; and
placing the second therapeutic agent in the intermediate region.

33. A medical device for at least partial implantation in a patient, comprising:
an outer tube member defining a lumen;
an inner tube member disposed in the lumen to define an annular space between the inner and outer tube members;
an intermediate member disposed in the annular space; and
a mixture of rifampin and minocycline evenly distributed throughout at least a portion of the intermediate member;
wherein minocycline is associated with at least one of the inner and outer tube members.
